(19) United States
(12) Patent Application Publication
(10) Pub. No.: US 2025/0152498 A1
(43) Pub. Date: May 15, 2025

(54) COMPOSITION

(71) Applicant: COSMETIC WARRIORS LIMITED, Poole Dorset (GB)

(72) Inventors: Mark CONSTANTINE, Poole Dorset (GB); Margaret Joan CONSTANTINE, Poole Dorset (GB); Helen Elizabeth AMBROSEN, Poole Dorset (GB); Wesley James BURRAGE, Poole Dorset (GB); Daisy Jaine EVANS, Poole Dorset (GB)

(21) Appl. No.: 18/726,366

(22) PCT Filed: Jan. 16, 2023
(86) PCT No.: PCT/GB2023/050070

§ 371 Date: Jul. 2, 2024

(30) Foreign Application Priority Data
Jan. 18, 2022 (GB) 2200582.1
Nov. 11, 2022 (GB) 2216893.4

(51) Int. Cl. A61K 8/9789; A61K 8/02; A61K 8/34; A61K 8/92; A61Q 5/06

(52) U.S. Cl. CPC A61K 8/9789; A61K 8/0216; A61K 8/345; A61K 8/922; A61Q 5/065; A61K 2800/5922; A61K 2800/80

(57) ABSTRACT

A solid cosmetic composition includes henna, a hard vegetable butter and a polyol. In addition a solid cosmetic composition includes henna, a hard vegetable butter and a herbal dye material selected from hibiscus, noni fruit, and combinations thereof.

### CROSS-REFERENCE TO RELATED APPLICATIONS

The present application is a National Stage Application of PCT International Application No.: PCT/GB2023/050070 filed on Jan. 16, 2023, which claims priority to GB Patent Application 2200582.1, filed in Great Britain on Jan. 18, 2022, and GB Patent Application 2216893.4, filed in Great Britain on Nov. 11, 2022, the disclosure of which are incorporated herein by reference in their entirety.

### FIELD OF THE INVENTION

The present invention relates to a solid cosmetic composition, a process for producing said solid cosmetic composition, a product prepared by the process, and a cosmetic method for using the solid cosmetic composition.

### BACKGROUND TO THE INVENTION

The present invention relates to solid cosmetic compositions, particularly those for use on the hair. Although the invention is primarily for use on hair, it may also be used on skin or other materials.

In the modern era, a high proportion of cosmetic products are typically provided to consumers in the form of liquid or pastes. For example, hair treatments and products, particularly cosmetic products, used for dyeing are conventionally supplied in containers made of a rigid or semi-rigid plastic material or of foil in the form of sachets. The container adds significantly to the cost of the products. From an environmental perspective, waste packaging is a significant problem, despite the availability of recycling.

Henna has traditionally been sold in the form of powders and pastes that typically require packaging in order for them to be transported and stored, and has been sold as a colouring in hair rinses, skin dyes, fabric dyes, and the like. As noted above, the required use of packaging is a disadvantage.

GB 2369995 describes a solid product comprising henna and cocoa butter. The amount of cocoa butter described as being preferred is 30% by weight.

The present invention seeks to provide a solid cosmetic product, which avoids the need to use external packaging, whilst providing the user with an improved hair dye product and improved delivery of henna.

### SUMMARY OF THE INVENTION

According to a first aspect of the invention, there is provided a solid cosmetic composition comprising (i) henna in an amount of from about 5% to about 75% by weight of the solid cosmetic composition; (ii) a hard vegetable butter in an amount of from about 5% to about 35% by weight of the solid cosmetic composition; and (iii) a polyol in an amount of from about 1% to about 35% by weight of the solid cosmetic composition.

According to a further aspect of the invention, there is provided a process for the production of a solid cosmetic composition according to the first aspect of the invention, the process comprising the steps of:
(a) combining:
   (i) henna,
   (ii) a hard vegetable butter, and
   (iii) a polyol; and
(b) allowing the resultant combination to solidify.
   According to a further aspect of the invention, there is provided a solid cosmetic composition obtained or obtainable by a process, the process comprising the steps of:
(a) combining:
   (i) henna,
   (ii) a hard vegetable butter, and
   (iii) a polyol; and
(b) allowing the resultant combination to solidify.

According to a further aspect of the invention, there is provided a solid cosmetic composition comprising (i) henna; (ii) a hard vegetable butter; and (iii) a herbal dye material selected from the group consisting of hibiscus, noni fruit, and combinations thereof.

According to a further aspect of the invention, there is provided a cosmetic method comprising contacting the hair of a user with the solid cosmetic composition as described herein.

The compositions of the invention including henna may be used as hair dyes in solid form. It is described in GB2369995 that the inclusion of cocoa butter is necessary in order to provide a hair dye product that has acceptable solidity and thus does not require packaging. The amounts of cocoa butter in previous products were relatively high, and typically between about 30 to 40%. It has now been surprisingly found by the present inventors that a lower amount of cocoa butter or other hard vegetable butter may provide a product that, when applied to the hair, results in shades of colour on the hair that have more depth and a more even coverage on the hair. The product may also provide an improved shade of colour when applied to hair. In particular, it was found that the dye materials included were able to deposit more pigment onto the strands of the hair.

However, reducing or removing hard vegetable butter was found to result in a composition that is not able to sustain its physical shape when unsupported by packaging. The hard vegetable butter was also found to provide benefits such as conditioning and moisture on the hair. The present inventors surprisingly found that the combination of a hard vegetable butter with a polyol, such as glycerol, provided a composition that has acceptable solidity and hardness such that it may be handled and transported easily without requiring any external packaging, whilst also providing a hair dye product that provides deeper shades of colour and a more even coverage on the hair than was previously possible. The resulting product was found to still provide a desirable conditioning effect and moisturising effect on the hair.

Without the presence of any polyol, it was found that the reduced cocoa butter products were drier and prone to crumbling, and more effort was required in order to press these products into the shape of a moulded brick. It was thus surprisingly found that the inclusion of a polyol improved the solidity of the product and reduced any effect of crumbling or cracking, whilst also improving the coverage of the product on the hair. It was found that the resulting products may provide improved coverage on grey hairs in particular.

It has also been surprisingly found that the combination of a hard vegetable butter and polyol (e.g. glycerol) provides a solid product that is easier to apply to the hair. The product has been found to be easier to break apart into pieces which may be subsequently melted such that the product can be applied to the hair. It has also been found that the product may melt more quickly.

Due in part to the solid form of the composition, it was also found that the composition may be resistant to microbiological growth without requiring the use of synthetic preservatives. The compositions may thus be regarded as “preservative-free”.

It has also been surprisingly found by the present inventors that the combination of a hard vegetable butter and henna with hibiscus and/or noni fruit may provide an enhanced colouring effect of the hair with deeper and richer tones being achieved as compared with a composition that does not include such herbal materials.

For ease of reference, these and further aspects of the present invention are now discussed under appropriate section headings. However, the teachings under each section are not necessarily limited to each particular section.

### DETAILED DESCRIPTION

### Composition

As described herein, in one aspect of the present invention, there is provided a solid cosmetic composition comprising: (i) henna in an amount of from about 5% to about 75% by weight of the solid cosmetic composition; (ii) a hard vegetable butter in an amount of from about 5% to about 35% by weight of the solid cosmetic composition; and (iii) a polyol in an amount of from about 1% to about 35% by weight of the solid cosmetic composition.

It will be understood by one skilled in the art that the nature of a cosmetic product means that the product is not edible. Thus, the present invention provides a non-edible solid cosmetic composition as defined herein.

Solid cosmetic products of the present invention are compositions which can substantially sustain their physical shape when unsupported by external means, e.g. packaging etc. Thus, they are considered to be solid, solid-like, in solid form or in solid-like form at room temperature. For the avoidance of doubt, the solid product is one which remains substantially solid at a temperature of up to 30° C.

By solid-like, it is understood that some materials are considered on a day to day basis to be solid, yet over an extremely long period of time, may alter in shape, e.g. amorphous materials such as glass etc. However, they are considered to be solid-like as, for the purpose they fulfil, they are solid.

As mentioned above, due to the solid form of the compositions of the present invention, external packaging is not required to maintain the shape of the composition. The solid form of the composition according to the invention thus enables a beneficial reduction in costs and reduction of environmental pollution to be achieved by avoiding the necessity of any packaging or container. In addition, by virtue of being in solid form, the composition can accept imprints or stamps that may be applied during production.

### Henna & Colouring Materials

As described herein, the solid cosmetic composition comprises henna. Henna is a dye prepared from the plant Lawsonia intermis, which may also be referred to as the “henna tree”. As used herein, the term “henna” refers only to material derived from the henna tree, and not to other skin and hair dyes, such as black henna and neutral henna, neither of which is derived from the henna tree. Henna is a colouring agent, which may be used to dye the skin or hair of a user, and which may also add shine to the user's hair.

Henna may be included in the compositions in powdered form. The henna may be provided in the form of milled leaves from the henna tree. The henna may preferably be red henna.

According to a first aspect of the invention, the solid cosmetic composition comprises henna in an amount of from about 5% to about 75% by weight of the solid cosmetic composition. In some preferred embodiments, the henna is present in an amount of from about 10% to about 75% by weight of the solid cosmetic composition, such as in an amount of from about 15% to about 75% by weight of the solid cosmetic composition. In some embodiments. the henna is present in an amount of from about 10% to about 70% by weight of the solid cosmetic composition, such as from about 15% to about 65% by weight of the solid cosmetic composition, such as from about 20% to about 60% by weight of the solid cosmetic composition. In some embodiments, the solid cosmetic composition comprises henna in an amount of from about 25% to about 70% by weight of the solid cosmetic composition, such as from about 30% to about 65% by weight of the solid cosmetic composition, such as from about 40% to about 65% by weight of the solid cosmetic composition. In some embodiments, the solid cosmetic composition comprises henna in an amount of from about 15% to about 50% by weight of the solid cosmetic composition, such as from about 15% to about 45% by weight of the solid cosmetic composition.

In some embodiments, the solid cosmetic composition comprises henna in an amount of from about 50% to about 65% by weight of the solid cosmetic composition. In some embodiments, the solid cosmetic composition comprises henna in an amount of from about 35% to about 50% by weight of the solid cosmetic composition. In some embodiments, the solid cosmetic composition comprises henna in an amount of from about 15% to about 30% by weight of the solid cosmetic composition, such as in an amount of from about 15% to about 25% by weight of the solid cosmetic composition.

The solid cosmetic composition may, in some embodiments, comprise other pigments. dyes or colouring materials in addition to the henna. Such additional pigments, dyes or colouring materials may be included to enhance the colour provided by the henna, to modify the overall colour provided to the hair by the composition, or a combination of the two. For example, an additional colouring material may be included to lighten or darken the shade of colour provided by the henna, or may be included simply to increase the intensity of the colour provided by the henna. Preferably, any additional dye, pigment or colouring material may be derived from natural sources.

In some embodiments, the solid cosmetic composition may comprise a herbal dye material. The herbal dye material is other than the henna. This means that the herbal dye material is a dye material derived from natural sources other than the henna tree (Lawsonia intermis). The herbal dye material may be a material that contains pigments capable of colouring the hair or skin of a user, and may thus also be referred to as a colouring material.

The herbal dye material may be any suitable naturally derived material capable of colouring the hair or skin of a user. The herbal dye material may be selected from a natural pigment, such as beetroot, chlorophyll, gardenia, blackberry, coffee, rose, caramel powder, grape, alfalfa, walnut hull, calendula, cocoa, green tea, hibiscus, kelp, olive, orange, parsley, pumpkin, spinach, spirulina, wheatgrass sources, sandalwood, saffron, safflower, turmeric, butterfly pea, carrot, tomato, indigo, Irish moss, nettle, noni fruit, rhubarb root, rosemary, chamomile, cassia obtava (also commonly referred to as “neutral henna”), spikenard, and mixtures thereof.

The herbal dye material may be selected from beetroot, chlorophyll, gardenia, blackberry, coffee, rose, caramel powder, grape, alfalfa, walnut hull, calendula, cocoa, green tea, hibiscus, kelp, olive, orange, parsley, pumpkin, spinach, spirulina, wheatgrass sources, sandalwood, saffron, safflower, turmeric, butterfly pea, carrot, tomato, indigo, Irish moss, nettle, noni fruit, rhubarb root, rosemary, chamomile, cassia obtava (also commonly referred to as “neutral henna”), spikenard, amla (Indian gooseberry), Bhrinhraj/maka (Eclipta alba or Eclipta prostrata), Kuth root, Valerian root, alkanet, blueberry, pomegranate, and combinations thereof.

Preferably, the herbal dye material may be selected from the group consisting of indigo, Irish moss, nettle, coffee (e.g. ground coffee), noni fruit, hibiscus, rhubarb root, rosemary, chamomile, cassia obtava, spikenard, amla (Indian gooseberry), Bhrinhraj/maka (Eclipta alba or Eclipta prostrata), Kuth root, Valerian root, alkanet, blueberry, pomegranate, and mixtures thereof. Preferably, the herbal dye material may be selected from the group consisting of indigo, Irish moss, nettle, coffee (e.g. ground coffee), noni fruit, hibiscus, rhubarb root, rosemary, chamomile, cassia obtava, spikenard, and mixtures thereof. The herbal dye material may be preferably added in powdered form.

In some embodiments, the herbal dye material may be present in an amount of from about 5% to about 60% by weight of the solid cosmetic composition, such as from about 5% to about 55% by weight of the solid cosmetic composition, such as from about 5% to about 50% by weight of the solid cosmetic composition. In some embodiments, the herbal dye material may be present in an amount of from about 10% to about 50% by weight of the solid cosmetic composition, such as from about 20% to about 45% by weight of the solid cosmetic composition, such as from about 25% to about 40% by weight of the solid cosmetic composition.

In some preferred embodiments, the total combined amount of henna and herbal dye material is from about 40% to about 94% by weight of the solid cosmetic composition. The “total combined amount” refers to the sum of the amount of henna and amount of herbal dye material that is other than henna. The total combined amount of henna and herbal dye material may preferably be from about 45% to about 90% by weight of the solid cosmetic composition, such as from about 50% to about 80% by weight of the solid cosmetic composition, such as from about 55% to about 75% by weight of the solid cosmetic composition, such as from about 60% to about 70% by weight of the solid cosmetic composition, such as from about 65% to about 70% by weight of the solid cosmetic composition. In some preferred embodiments, the total combined amount of henna and herbal dye material is from about 40% to about 80% by weight of the solid cosmetic composition.

In some embodiments, the weight ratio of henna to herbal dye material may be from about 15:1 to about 1:5, such as from about 10:1 to about 1:3, such as from about 5:1 to about 1:3. Depending on the shade of hair colour desired, the weight ratio of henna to herbal dye material may be from about 2:1 to about 1:3, such as from about 1.5:1 to about 2:1.

In some preferred embodiments, the solid cosmetic composition comprises a herbal dye material, where the herbal dye material comprises at least indigo. Indigo herb may be derived from the plant Indigofera tinctoria, and is a blue dye. It has been found that indigo herb may impart an inky black colour to hair. When combined with henna, the inventors found that indigo may provide warm colour tones and an even coverage of colour on the hair. True indigo (Indigofera tinctoria) is a shrub from the bean family that is cultivated under tropical and subtropical climates. Indigo powder may be obtained by grinding the leaves of the plant after they've been dried. Where present, indigo (preferably in the form of a powder) may be included in an amount of from about 5% to about 50% by weight of the solid cosmetic composition, for example from about 5% to about 20% by weight or from about 25% to about 45% by weight of the solid cosmetic composition.

Preferably, the solid cosmetic composition comprises a herbal dye material, where the herbal dye material comprises at least indigo, noni fruit, hibiscus, spikenard or combinations thereof. Preferably, the solid cosmetic composition comprises a herbal dye material, where the herbal dye material comprises at least indigo powder, noni fruit powder, hibiscus powder, spikenard or combinations thereof.

Preferably, the solid cosmetic composition comprises a herbal dye material, where the herbal dye material comprises at least noni fruit, hibiscus, or combinations thereof. Preferably, the solid cosmetic composition comprises a herbal dye material, where the herbal dye material comprises at least noni fruit powder, hibiscus powder, or combinations thereof. It has been surprisingly found by the present inventors that the inclusion of at least one of noni fruit and hibiscus (preferably in powdered form) may improve the colour imparted to the skin or hair by the solid cosmetic composition. For example, henna can sometimes result in an orange tone when applied to the hair, and it has been found that noni fruit and/or hibiscus may reduce harsher orange tones. In particular, it was found that the inclusion of at least noni fruit and/or hibiscus in combination with henna resulted in a product that provided improved colour tones than products where such herbal dye materials were not included. The resulting colour on the hair may be deeper and have cooler tones.

In some embodiments, the solid cosmetic composition may comprise at least one of noni fruit and hibiscus in an amount of from 0.1% to about 15% by weight of the solid cosmetic composition, such as from about 0.5% to about 10% by weight of the solid cosmetic composition, such as from about 1% to about 9% by weight of the solid cosmetic composition, such as from about 2% to about 8% by weight of the solid cosmetic composition. These amounts refer to the total combined amount of noni fruit and/or hibiscus, optionally in powdered form.

In some embodiments, the herbal dye material comprises at least hibiscus powder. In some embodiments, the herbal dye material is hibiscus powder. The herbal dye material may be or comprise hibiscus powder in an amount of from about 0.1% to about 15% by weight of the solid cosmetic composition, such as from about 0.5% to about 12.5% by weight of the solid cosmetic composition, such as from about 1% to about 10% by weight of the solid cosmetic composition, such as from about 5% to about 10% by weight of the solid cosmetic composition.

Hibiscus is a genus of flowering plants in the mallow family, Malvaceae. Hibiscus powder may be obtained from the leaves and/or flowers of the hibiscus plant. Preferably the hibiscus powder is obtained from the flowers of the hibiscus plant. Hibiscus powder may provide, not only a colouring effect for the skin or hair of the user, but also a gentle cleansing and conditioning effect. Hibiscus powder may also increase the shine of the hair, and may improve the colour tones of hair when combined with henna.

In some embodiments, the herbal dye material comprises at least noni fruit powder. In some embodiments, the herbal dye material is noni fruit powder. The herbal dye material may be or comprise noni fruit powder in an amount of from about 0.1% to about 15% by weight of the solid cosmetic composition, such as from about 0.5% to about 10% by weight of the solid cosmetic composition, such as from about 1% to about 5% by weight of the solid cosmetic composition, such as from about 2% to about 5% by weight of the solid cosmetic composition.

Noni is an evergreen tree referred to as Morinda citrifolia, which includes large leaves and yellow fruits. Noni fruit powder may be derived from the dried ripe or unripe fruits from the noni tree, and is rich in vitamins, mineral and antioxidants. Noni fruit powder may enhance the shine on the hair as well as improving the colour tones of hair when combined with henna.

### Hard Vegetable Butter

As described herein, the solid cosmetic composition comprises a hard vegetable butter. As used herein, the term “vegetable butter” refers to triglycerides which are derived from plant or vegetable sources and which are found to be solid (including solid-like, discussed above) at normal usage temperatures. A vegetable butter is thus a triglyceride which is substantially solid at room temperature. Vegetable butters typically have the consistency of a butter. It will be appreciated however that it is not a requirement that the vegetable butter have a solid fat content of 100% at normal usage temperatures. In a preferred aspect the solid fat has a solid fat content of at least 70%, preferably at least 80%, preferably at least 90%, preferably at least 95%, preferably at least 98%, preferably at least 99% at 25° C.

The term “hard” vegetable butter refers to a vegetable butter that comprises a majority amount of saturated fatty acids. In particular, hard vegetable butters are typically those that include greater than 60 wt % saturated fatty acids based on the total fatty acids of the high saturated vegetable butter. Hard vegetable butters may therefore also be referred to as high saturated vegetable butters.

In some embodiments, the hard vegetable butter may be selected from the group consisting of cocoa butter, illipe butter, murumuru butter, kokum butter, coconut oil, palm kernel oil, and mixtures thereof. While coconut oil and palm kernel oil may typically be referred to as ‘oils’, they can be regarded as hard vegetable butters since they are solid at room temperature and have a high saturated fat content.

In some embodiments, the hard vegetable butter may be selected from the group consisting of cocoa butter, illipe butter, murumuru butter, kokum butter, coconut oil, and mixtures thereof.

In some preferred embodiments, the hard vegetable butter comprises as least cocoa butter. In some preferred embodiments, the hard vegetable butter is cocoa butter.

In some embodiments, the hard vegetable butter (e.g. cocoa butter) may be present in an amount of from about 5% to about 35% by weight of the solid cosmetic composition.

In some embodiments, the hard vegetable butter (e.g. cocoa butter) may be present in an amount of from about 5% to about 30% by weight of the solid cosmetic composition, such as from about 5% to about 25% by weight of the solid cosmetic composition, such as from about 5% to about 20% by weight of the solid cosmetic composition, such as from about 10% to about 20% by weight of the solid cosmetic composition, such as from about 10 to about 15% by weight of the solid cosmetic composition.

It may be preferred that the hard vegetable butter (e.g. cocoa butter) is present in an amount of at least 5% of the solid cosmetic composition to provide sufficient solidity to the composition that it can be stored for long periods of time without requiring packaging. However, it has been surprisingly found that the inclusion of very high amounts of cocoa butter may result in a more uneven coverage of colour on the hair when the composition is applied to the hair, and may also provide a more muted colour. It was surprisingly found that, when lower amounts of hard vegetable butter than previously used were included in the composition, the resulting colour on the hair was a more vibrant shade with added depth and coverage. Therefore, the amount of hard vegetable butter in the solid cosmetic composition is no greater than about 35%, such as no greater than about 30% by weight of the solid cosmetic composition. In some preferred embodiments, the amount of hard vegetable butter in the solid cosmetic composition may be no greater than about 25%, and preferably no greater than about 20% or no greater than about 15% by weight of the solid cosmetic composition.

In some embodiments, the solid cosmetic composition comprises hard vegetable butter (e.g. cocoa butter) in an amount of from about 11% to about 19% by weight, such as from about 12% to about 18% by weight, such as from about 13% to about 17% by weight, such as from about 14% to about 16% by weight, such as approximately 15% by weight of the solid cosmetic composition.

### Polyol

As described herein, the solid cosmetic composition comprises a polyol. The term “polyol” refers to an organic compound that comprises more than one hydroxyl groups.

In some embodiments, the polyol may be or comprise a sugar alcohol. The term “sugar alcohol” refers to a class of low molecular weight polyols that may typically be derived from sugars, e.g. via hydrogenation. Sugar alcohols may include one hydroxyl group attached directly or indirectly to each carbon atom in their chain. Examples of sugar alcohols include ethylene glycol, glycerol, erythritol, threitol, arabitol, xylitol, ribitol, mannitol, sorbitol, galactitol, inositol, isomalt, maltitol and lactitol.

Alternatively or in addition, the polyol may be or comprise propylene glycol (propan-1,2-diol) or propan-1,3-diol.

In some embodiments, the polyol may be selected from the group consisting of ethylene glycol, glycerol, erythritol, threitol, arabitol, xylitol, ribitol, mannitol, sorbitol, galactitol, inositol, isomalt, maltitol, lactitol, propylene glycol, propan-1,3,-diol and mixtures thereof. In some embodiments, the polyol may be selected from the group consisting of glycerol, propylene glycol, propan-1,3-diol, sorbitol, xylitol, isomalt, erythritol, lactitol, mannitol, maltitol, and mixtures thereof. In some embodiments, the polyol may be selected from the group consisting of glycerol, sorbitol, xylitol, isomalt, mannitol, and mixtures thereof. In some embodiments, the polyol may be selected from the group consisting of glycerol, sorbitol and mixtures thereof.

In some preferred embodiments, the solid cosmetic composition may be free from or substantially free from (i.e. include less than 1% by weight of) propylene glycol. As such, in some embodiments, the polyol may not be or comprise propylene glycol.

In some preferred embodiments, the polyol comprises at least glycerol. Preferably, the polyol is glycerol.

In some embodiments, the solid cosmetic composition comprises a polyol (e.g. glycerol) in an amount of from about 0.1% to about 50% by weight of the solid cosmetic composition, such as from about 0.5% to about 40% by weight of the solid cosmetic composition.

In preferred embodiments, the solid cosmetic composition comprises a polyol (e.g. glycerol) in an amount of from about 1% to about 35% by weight of the solid cosmetic composition. In some embodiments, the solid cosmetic composition comprises a polyol (e.g. glycerol) in an amount of from about 5% to about 30% by weight of the solid cosmetic composition, such as from about 10% to about 25% by weight of the solid cosmetic composition, such as from about 10% to about 20% by weight of the solid cosmetic composition. In some embodiments, the solid cosmetic composition may comprise a polyol (e.g. glycerol) in an amount of from about 5% to about 20% by weight of the solid cosmetic composition.

### Further Components

In addition to the above, the cosmetic composition may further comprise one or more cosmetically acceptable additives. The person skilled in the art is aware of a range of cosmetically acceptable additives which are suitable for incorporation into such compositions.

In some embodiments, the one or more cosmetically acceptable additives are selected from a wax, starch, binder, filler, clay, opacifier, UV absorbing material, UV reflecting material, exfoliating material (such as salt and/or sugar), essential oil, vitamin, perfume, fragrance, colouring, soft vegetable butter, vegetable oil, honey, fruit, fruit juice, fruit and/or herb extract, protein, decorative item, emollient, effervescent component (e.g. a combination of an acid and a base that reacts to cause effervescence in the presence of water), surfactant, humectant, conditioning agent, acidifying agent, a base and mixtures thereof. In some embodiments, the one or more cosmetically acceptable additives are selected from the group consisting of a fruit juice, binder, filler, exfoliating material, essential oil, vitamin, perfume, fragrance, colouring agent, decorative item, effervescent component, surfactant, humectant, conditioning agent, wax, soft vegetable butter, vegetable oil, acidifying agent, a base and mixtures thereof.

Preferably, the one or more cosmetically acceptable additives may be selected from a binder, filler, exfoliating material, essential oil, vitamin, perfume, fragrance, colouring agent, decorative item, effervescent component, surfactant, humectant, conditioning agent, acidifying agent, a base, and mixtures thereof.

In some embodiments, the solid cosmetic composition further comprises a fruit juice. In some embodiments, where present, the fruit juice may be included in an amount of from about 0.01% to about 15% by weight of the solid cosmetic composition, such as in an amount of from about 0.1% to about 10% by weight of the solid cosmetic composition, such as in an amount of from about 0.5% to about 7.5% by weight of the solid cosmetic composition, such as in an amount of from about 1% to about 5% by weight of the solid cosmetic composition. In some preferred embodiments, the fruit juice may be present in an amount of from about 0.1% to about 8% by weight of the solid cosmetic composition.

It has been found that the inclusion of a fruit juice may enhance the effect of colouring of the hair, with some fruit juices being found to provide lighter tones to the hair or being found to increase the shine of the hair. For example, lemon juice has been found to lighten the hair, and noni juice has been found to add colour to the hair. Any suitable fruit juice may be included. In some embodiments, the fruit juice may be selected from the group consisting of lemon juice, lime juice, grapefruit juice, yuzu juice, noni fruit juice, and mixtures thereof. In some embodiments, the fruit juice may be selected from the group consisting of lemon juice, noni fruit juice, and mixtures thereof. In some embodiments, the fruit juice is or comprises lemon juice. In some embodiments, the fruit juice is or comprises noni fruit juice.

In addition or as an alternative to fruit juice, the solid cosmetic composition may in some embodiments further comprise fruit and/or fruit extract. The fruit may be any suitable fruit. Preferably, the fruit may be selected from orange, mango, papaya, strawberry, banana, kiwi, apple, cherry, pineapple, raspberry, blueberry, blackberry, peach, nectarine, and mixtures thereof.

Other acidifying agents may be included in place of or in addition to fruit, fruit juice and/or fruit extract. For example, organic acidifying agents such as citric acid, tartaric acid, potassium bitartrate (cream of tartar) or mixtures thereof may be included in the composition. Where present, such acidifying agent may be present in an amount of from about 0.01% to about 15% by weight of the solid cosmetic composition.

Fragrance may be added to the product to make the experience of using the present composition more pleasant. For example, combining essential oils such as lavender or rose absolute into fragrances for the invention ensures the user has a pleasant experience.

In some embodiments, the cosmetic composition thus further comprises a perfume or a fragrance. The amount of fragrance is preferably from about 0.01% to about 10% by weight of the total composition, such as from about 0.1% to about 5% by weight of the total composition, such as from about 0.5% to about 5% by weight of the total composition, such as from about 0.5% to about 2.5% by weight of the total composition.

The essential oils may be selected based on the fragrance desired, skin type to be treated and other effects desired based on the well-known properties of essential oils. The addition of essential oils, when taken into the nose, is known to alter mood. For example, essential oils are known to create effects of drowsiness or stimulating the senses. Many well documented effects can be achieved by the use of essential oils.

In some embodiments, the one or more essential oils present in the product are selected from tarragon, lemon myrtle, jasmin, ylang ylang, labdanum, lemongrass, rose otto, grapefruit, patchouli, rosemary, armois, lemon, neroli, sweet violet, lavender, orange 50 fold, vanilla, peppermint, benzoin, hydrangea, litsea cubeba, cardamon, tonka, and chamomile. In some embodiments, the one or more essential oils present in the product are selected from tarragon, lemon myrtle, labdunum, and lemon.

Vitamins, particularly B, C and E are very beneficial for the skin and/or hair. Vitamin rich ingredients such as Wheatgerm oil can also be used to deliver vitamins on to the skin. In a one embodiment, the vitamins are selected from vitamin B, vitamin C, vitamin E and mixtures thereof. It will be appreciated by one skilled in the art that the vitamin may be provided from any suitable source. For example the vitamin(s) may be provided from a synthetic source or from incorporation into the product of a material, such as a natural material, that has a high vitamin content.

An exfoliating material may also be added to the composition to enhance the effect on the user's skin and/or hair. For example, in some embodiments, the solid cosmetic composition comprises salt (i.e. sodium chloride). Where present, salt may be included in an amount of from about 0.01% to about 10%, such as from about 0.1% to about 5%, and such as from about 0.5% to about 2.5%.

In some embodiments, the solid cosmetic composition further comprises a colouring agent. The colouring agent may be other than henna or herbal dye material described hereinabove. The colouring agent may be any pigment and/or dye suitable for imparting colour to the composition itself. In some embodiments, the solid cosmetic composition may comprise a colouring agent in an amount of from about 0.001% to about 5% by weight of the total composition.

In some embodiments, the solid cosmetic composition further comprises a protein. The protein may be selected from the group consisting of tofu, banana, soya, soya lecithin, hydrolysed wheat protein, and mixtures thereof. When present, the protein may be included in the solid cosmetic composition in an amount of from about 0.01% to about 10% by weight of the solid cosmetic composition, such as in an amount of from about 0.1% to about 5% by weight of the solid cosmetic composition.

The solid cosmetic composition may further comprise a surfactant. Surfactants may be useful where the intended use of the product is as a cleansing agent as well as a hair dye and/or skin colourant or dye. Where present, the surfactant may be selected from the group consisting of anionic surfactant, amphoteric surfactant, cationic surfactant, non-ionic surfactant and mixtures thereof. For example, a surfactant may be selected from the group consisting of sodium lauryl sulfate, sodium laureth sulfate, sodium dodecyl sulfate, sodium cocosulfate, ammonium lauryl sulfate, ammonium laureth sulfate, sodium myreth sulfate, sodium lauroyl sarcosinate, disodium laureth sulfosuccinate, disodium lauryl sulfosuccinate, sodium lauryl sulfoacetate, sodium cocoyl isethionate, sodium olefin sulfonate, lauryl betaine, cocamidopropyl betaine, trimethyl glycine betaine, sodium cocoamphoacetate, disodium cocoamphodiacetate, sodium lauroamphoacetate, disodium lauroamphodiacetate, cocoamidopropyl hydroxysultaine, sodium lauroyl sarcosine, cetrimonium chloride and mixtures thereof. In some embodiments, the solid cosmetic composition may include a surfactant selected from sodium lauryl sulfate, sodium laureth sulfate and/or cocamidopropyl betaine.

In some embodiments, a conditioning agent may be included in the composition, such as a hair conditioning agent. Examples of hair conditioning agents may include behenzyl trimethyl ammonium chloride, cationic guar gum, cetrimonium bromide or chloride, other quaternary cationic surfactants, emulsifying waxes, sodium polynaphthalene sulfonate, stearamidopropyl dimethylamine (aka N-[3-(dimethylamino)propyl] octadecanamide) and mixtures thereof. Suitable emulsifying waxes may include cetearyl alcohol (aka cetostearyl alcohol or cetylstearyl alcohol), stearic acid, glyceryl stearate, a combination of cetearyl alcohol and sodium lauryl sulphate (SLS), and mixtures thereof.

The solid cosmetic composition may further comprise a soft vegetable butter, a vegetable oil and/or a wax in addition to the hard vegetable butter. However, it is preferred that the composition does not include any butters, oils or waxes that are additional to the hard vegetable butter in significant amounts, and preferably is free from any such additional butters, oils or waxes. Preferably, the total combined amount of soft vegetable butter, vegetable oil and wax is no greater than 10%, preferably no greater than 5%, and more preferably no greater than 1% by weight of the solid cosmetic composition. In some embodiments, the solid cosmetic composition is free from each of soft vegetable butter, vegetable oil and wax. By “soft vegetable butter” is meant a low saturated vegetable butter that includes less than 60 wt % saturated fatty acids based on the total fatty acids of the low saturated vegetable butter. “Vegetable oil” refers to a vegetable triglyceride that is liquid at room temperature.

In some embodiments, the hard vegetable butter is the only source of fat or triglyceride present in the composition.

### Total Composition

In some embodiments, the weight ratio of hard vegetable butter (e.g. cocoa butter) to polyol (e.g. glycerol) is from about 10:1 to about 1:10, such as from about 5:1 to about 1:5, such as from about 4:1 to about 1:4, such as from about 2:1 to about 1:2, such as approximately 1:1. In some preferred embodiments, the weight ratio of hard vegetable butter (e.g. cocoa butter) to polyol (e.g. glycerol) may be from about 1:2 to about 1:1. It was found by the present inventors that, when the hard vegetable butter and polyol are provided in such weight ratios, the composition has acceptable solidity and hardness that it may be handled and transported easily and also have a good feel in the hand of the user, whilst also providing an improved and desirable colour intensity on the hair and/or skin. For example, if the weight ratio of hard vegetable butter to polyol agent is too low, then the product may not be sufficiently solid, and also may become tacky or sticky over time (e.g. during storage). If the weight ratio of hard vegetable butter to polyol is too high, then the product may not provide as intense a colour on the hair or as even a coverage on the hair of the user and thus may result in a less desirable finish.

In some embodiments, the weight ratio of henna to hard vegetable butter (e.g. cocoa butter) is from about 1:1 to about 10:1, such as from about 1:1 to about 1:5. It has been surprisingly found that reducing the relative amount of hard vegetable butter in the formulation may provide an enhanced colour when the product is applied to the hair. For example, the coverage of colour on the hair may be improved, and the intensity of colour on the hair may be increased.

In some embodiments, the solid cosmetic composition comprises:
(i) henna in an amount of from about 5% to about 75% by weight of the solid cosmetic composition;
(ii) cocoa butter in an amount of from about 5% to about 35% by weight of the solid cosmetic composition; and
(iii) glycerol in an amount of from about 1% to about 35% by weight of the solid cosmetic composition.
   In some embodiments, the solid cosmetic composition comprises:
(i) henna in an amount of from about 5% to about 75% by weight of the solid cosmetic composition;
(ii) a hard vegetable butter in an amount of from about 5% to about 35% by weight of the solid cosmetic composition;
(iii) a polyol in an amount of from about 1% to about 35% by weight of the solid cosmetic composition; and
(iv) a herbal dye material selected from the group consisting of indigo powder, Irish moss powder, nettle powder, ground coffee, noni fruit powder, hibiscus powder, rhubarb root powder, chamomile powder, cassia obtava, rosemary powder, spikenard, and mixtures thereof,
wherein the total combined amount of henna and herbal dye material is from about 40% to about 80% by weight of the solid cosmetic composition, and preferably from about 50% to about 80% by weight of the solid cosmetic composition.

In some preferred embodiments, the composition comprises water in a total amount of no greater than about 15% by weight of the solid cosmetic composition, such as in a total amount of no greater than about 10% by weight of the solid cosmetic composition, such as in a total amount of no greater than about 5% by weight of the solid cosmetic composition, such as in a total amount of no greater than about 1% by weight of the solid cosmetic composition, such as in a total amount of no greater than 0.5% by weight of the solid cosmetic composition, such as in a total amount of no greater than 0.1% by weight of the solid cosmetic composition.

Preferably, the solid cosmetic composition is suitable for use in dyeing the hair and/or skin of the user. Preferably the solid cosmetic composition is a solid hair dye.

The above ranges provide preferred amounts of each of the components. Each of these ranges may be taken alone or combined with one or more other component ranges to provide a preferred aspect of the invention.

### Process

As described herein, there is provided a process for the production of a solid cosmetic composition as defined herein, the process comprising the steps of:
(a) combining:
   (i) henna,
   (ii) a hard vegetable butter, and
   (iii) a polyol; and
(b) allowing the resultant combination to solidify.

As described herein, the solid cosmetic composition may further comprise one or more cosmetically acceptable additives. In some embodiments, the process further comprises the step of combining with the mixture of step (a) one or more cosmetically acceptable additives as defined herein. For example, in some embodiments, the process further comprises adding fruit juice, herbal dye material or combinations thereof before, during or after step (a). The herbal dye material may be added in powdered form.

For example, any additives present may be added to a container together with the henna, vegetable butter and polyol all at roughly the same time (i.e. during step (a)). Alternatively, any additives present may be added after the henna, vegetable butter and polyol have already been combined (i.e. after step (a)), but before allowing the resultant combination to solidify.

In some specific embodiments where the composition comprises a herbal dye material, the henna and herbal dye material may be combined (e.g. via mixing). Where the herbal dye material and henna are provided as powders, the powders may be added together (e.g. via sifting). Separately, the hard vegetable butter and polyol may be combined (e.g. via mixing). The combination of herbal dye material and henna may then be added slowly to the combination of hard vegetable butter and polyol whilst mixing.

Step (b) may involve a passive step of solidification, whereby the mixture is simply left at room temperature and exposed to air at ambient pressure and humidity for a period of time until the mixture has solidified. For example, the mixture may be left for a period of at least about 1 hour to set, and preferably at least 12 hours or 24 hours.

In some embodiments, step (b) may involve a step of active solidification, which may comprise modifying the ambient temperature, pressure or humidity in order to increase the rate of solidification.

Step (b) may comprise introducing the resultant combination from step (a) into a mould before allowing the combination to solidify. In this way, a moulded product may be formed. Optionally, the mixture may be pressed when adding the mixture to the mould or during the drying process, such that a product in the form of a moulded bar or brick may be obtained.

In some embodiments, the resultant combination from step (a) may be in the form of a paste, which is then hardened to provide a solid cosmetic composition.

The shape of the solid compositions of the present invention is not limited. It may be that the solid compositions are provided with a shape that would be aesthetically pleasing and/or which aids in the use of the product. For example, it may be that the solid product is produced in such a manner so that it solidifies in a shape which is ergonomically acceptable to the user. Therefore, in some embodiments of the process of the present invention, the mixture resulting after step (a) is poured and/or pressed into a mould, allowed to solidify, and then turned out to produce the solid product.

The compositions in solid form may include some decoration or instruction on their surface, which may be applied before, during or after the step (b) of the above process. For example, the products may be stamped with a design (e.g. words or picture) before, during or after solidification. For example, the products may be stamped during solidification but before the product has set completely. In some embodiments, the compositions may be etched or have some image or word affixed to their surface.

The present invention also provides a solid cosmetic composition obtained or obtainable by a process as described herein.

### Further Aspects—Composition

According to a further aspect of the invention, there is provided a solid cosmetic composition comprising (i) henna; (ii) a hard vegetable butter; and (iii) a herbal dye material selected from the group consisting of hibiscus, noni fruit, amla (Indian gooseberry), Bhrinhraj/maka (Eclipta alba or Eclipta prostrata), spikenard, Kuth root, Valerian root, alkanet, blueberry, pomegranate, and combinations thereof.

According to a further aspect of the invention, there is provided a solid cosmetic composition comprising (i) henna; (ii) a hard vegetable butter; and (iii) a herbal dye material selected from the group consisting of hibiscus, noni fruit, and combinations thereof.

All of the disclosure—and in particular the ranges—detailed above with respect to the first aspect may equally apply to the composition according to these further aspects. For example, the hard vegetable butter may preferably be or comprise cocoa butter.

Thus, in some embodiments, the solid cosmetic composition comprises:
(i) henna in an amount of from about 5% to about 75% by weight of the solid cosmetic composition;
(ii) a hard vegetable butter in an amount of from about 5% to about 35% by weight of the solid cosmetic composition; and
(iii) a herbal dye material selected from the group consisting of hibiscus, noni fruit, and combinations thereof.

Preferably, the hibiscus and/or noni fruit is included in powdered form. Thus the composition may preferably comprise a herbal dye material selected from the group consisting of hibiscus powder, noni fruit powder, and combinations thereof.

The hibiscus (e.g. powder) and/or noni fruit (e.g. powder) may be included in a total amount of from about 0.1% to about 15% by weight of the solid cosmetic composition, such as from about 0.5% to about 12.5% by weight of the solid cosmetic composition. In some embodiments, the solid cosmetic composition may comprise at least one of noni fruit and hibiscus in an amount of from 0.1% to about 15% by weight of the solid cosmetic composition, such as from about 0.5% to about 10% by weight of the solid cosmetic composition, such as from about 1% to about 9% by weight of the solid cosmetic composition, such as from about 2% to about 8% by weight of the solid cosmetic composition. These amounts refer to the total combined amount of noni fruit and/or hibiscus, optionally in powdered form.

In some embodiments, the solid cosmetic composition comprises at least hibiscus powder, where the hibiscus powder is present in an amount of from about 1% to about 10% by weight of the solid cosmetic composition, such as from about 5% to about 10% by weight of the solid cosmetic composition. The herbal dye material may be or comprise hibiscus powder in an amount of from about 0.1% to about 15% by weight of the solid cosmetic composition, such as from about 0.5% to about 12.5% by weight of the solid cosmetic composition, such as from about 1% to about 10% by weight of the solid cosmetic composition, such as from about 5% to about 10% by weight of the solid cosmetic composition.

In some embodiments, the solid cosmetic composition comprises at least noni fruit powder, where the noni fruit powder is present in an amount of from about 1% to about 5% by weight of the solid cosmetic composition, such as from about 2% to about 5% by weight of the solid cosmetic composition. The herbal dye material may be or comprise noni fruit powder in an amount of from about 0.1% to about 15% by weight of the solid cosmetic composition, such as from about 0.5% to about 10% by weight of the solid cosmetic composition, such as from about 1% to about 5% by weight of the solid cosmetic composition, such as from about 2% to about 5% by weight of the solid cosmetic composition.

Where present, noni fruit may be included in the form of noni fruit powder, noni fruit juice, or combinations thereof. In some embodiments, the total amount of noni fruit (i.e. total amount of noni fruit powder and noni fruit juice) is from about 0.1% to about 15% by weight of the solid cosmetic composition, such as from about 0.5% to about 10% by weight of the solid cosmetic composition, such as from about 1% to about 5% by weight of the solid cosmetic composition, such as from about 2% to about 5% by weight of the solid cosmetic composition. In some embodiments, the total amount of noni fruit is from about 1% to about 7.5% by weight of the solid cosmetic composition

A solid cosmetic composition according to this aspect may also include herbal dye materials in addition to hibiscus and/or noni fruit, provided that at least one of hibiscus and/or noni fruit is also present. Examples of other herbal dye materials that may be included are detailed hereinabove, and include a natural pigment, such as beetroot, chlorophyll, gardenia, blackberry, coffee, rose, caramel powder, grape, alfalfa, walnut hull, calendula, cocoa, green tea, kelp, olive, orange, parsley, pumpkin, spinach, spirulina, wheatgrass sources, sandalwood, saffron, safflower, turmeric, butterfly pea, carrot, tomato, indigo, Irish moss, nettle, rhubarb root, rosemary, chamomile, cassia obtava, spikenard, and mixtures thereof. The additional herbal dye material may be selected from beetroot, chlorophyll, gardenia, blackberry, coffee, rose, caramel powder, grape, alfalfa, walnut hull, calendula, cocoa, green tea, kelp, olive, orange, parsley, pumpkin, spinach, spirulina, wheatgrass sources, sandalwood, saffron, safflower, turmeric, butterfly pea, carrot, tomato, indigo, Irish moss, nettle, rhubarb root, rosemary, chamomile, cassia obtava (also commonly referred to as “neutral henna”), spikenard, amla (Indian gooseberry), Bhrinhraj/maka (Eclipta alba or Eclipta prostrata), Kuth root, Valerian root, alkanet, blueberry, pomegranate, and combinations thereof.

Preferably, the additional herbal dye material may be selected from the group consisting of indigo, Irish moss, nettle, coffee (e.g. ground coffee), rhubarb root, rosemary, chamomile, cassia obtava, spikenard, amla (Indian gooseberry), Bhrinhraj/maka (Eclipta alba or Eclipta prostrata), Kuth root, Valerian root, alkanet, blueberry, pomegranate, and mixtures thereof.

Preferably, the additional herbal dye material may be selected from the group consisting of indigo, Irish moss, nettle, coffee (e.g. ground coffee), rhubarb root, rosemary, chamomile, cassia obtava, spikenard, and mixtures thereof. The additional herbal dye material may be preferably added in powdered form.

In some embodiments, the additional herbal dye material may be present in an amount of from about 0.1% to about 60% by weight of the solid cosmetic composition, such as from about 0.1% to about 50% by weight of the solid cosmetic composition, such as from about 0.1% to about 40% by weight of the solid cosmetic composition. In some embodiments, the additional herbal dye material may be present in an amount of from about 5% to about 60% by weight of the solid cosmetic composition, such as from about 10% to about 55% by weight of the solid cosmetic composition, such as from about 15% to about 50% by weight of the solid cosmetic composition. In some embodiments, the additional herbal dye material may be present in an amount of from about 10% to about 50% by weight of the solid cosmetic composition, such as from about 20% to about 45% by weight of the solid cosmetic composition, such as from about 25% to about 40% by weight of the solid cosmetic composition.

In some preferred embodiments, the henna is present in an amount of from about 5% by weight to about 75% by weight of the solid cosmetic composition, such as from about 10% to about 75% by weight of the solid cosmetic composition, such as in an amount of from about 15% to about 75% by weight of the solid cosmetic composition. In some embodiments, the henna is present in an amount of from about 10% to about 70% by weight of the solid cosmetic composition, such as from about 15% to about 65% by weight of the solid cosmetic composition, such as from about 20% to about 60% by weight of the solid cosmetic composition. In some embodiments, the solid cosmetic composition comprises henna in an amount of from about 25% to about 70% by weight of the solid cosmetic composition, such as from about 30% to about 65% by weight of the solid cosmetic composition, such as from about 40% to about 65% by weight of the solid cosmetic composition. In some embodiments, the solid cosmetic composition comprises henna in an amount of from about 15% to about 50% by weight of the solid cosmetic composition, such as from about 15% to about 45% by weight of the solid cosmetic composition.

In some embodiments, the solid cosmetic composition comprises henna in an amount of from about 50% to about 65% by weight of the solid cosmetic composition. In some embodiments, the solid cosmetic composition comprises henna in an amount of from about 35% to about 50% by weight of the solid cosmetic composition. In some embodiments, the solid cosmetic composition comprises henna in an amount of from about 15% to about 30% by weight of the solid cosmetic composition, such as in an amount of from about 15% to about 25% by weight of the solid cosmetic composition.

In some embodiments, the herbal dye material (including hibiscus and/or noni fruit in combination with any additional herbal dye material) may be present in an amount of from about 5% to about 60% by weight of the solid cosmetic composition, such as from about 5% to about 55% by weight of the solid cosmetic composition, such as from about 5% to about 50% by weight of the solid cosmetic composition. In some embodiments, the herbal dye material may be present in an amount of from about 10% to about 50% by weight of the solid cosmetic composition, such as from about 20% to about 45% by weight of the solid cosmetic composition, such as from about 25% to about 40% by weight of the solid cosmetic composition.

In some preferred embodiments, the total combined amount of henna and herbal dye material (including hibiscus and/or noni fruit in combination with any additional herbal dye material) is from about 40% to about 94% by weight of the solid cosmetic composition. The “total combined amount” refers to the sum of the amount of henna and amount of herbal dye material that is other than henna. The total combined amount of henna and herbal dye material may preferably be from about 45% to about 90% by weight of the solid cosmetic composition, such as from about 50% to about 80% by weight of the solid cosmetic composition, such as from about 55% to about 75% by weight of the solid cosmetic composition, such as from about 60% to about 70% by weight of the solid cosmetic composition, such as from about 65% to about 70% by weight of the solid cosmetic composition. In some preferred embodiments, the total combined amount of henna and herbal dye material is from about 40% to about 80% by weight of the solid cosmetic composition.

In some embodiments, the weight ratio of henna to herbal dye material may be from about 15:1 to about 1:5, such as from about 10:1 to about 1:3, such as from about 5:1 to about 1:3. Depending on the shade of hair colour desired, the weight ratio of henna to herbal dye material may be from about 2:1 to about 1:3, such as from about 1.5:1 to about 2:1.

In some embodiments, the additional herbal dye material comprises indigo (preferably in the form of a powder). Indigo may be included in an amount of from about 5% to about 50% by weight of the solid cosmetic composition, for example from about 5% to about 20% by weight or from about 25% to about 45% by weight of the solid cosmetic composition.

The solid cosmetic composition further comprises a hard vegetable butter. All of the disclosure provided hereinabove with respect to the first aspect equally applies to the hard vegetable butter of this further aspect.

In some embodiments, the hard vegetable butter may be selected from the group consisting of cocoa butter, illipe butter, murumuru butter, kokum butter, coconut oil, palm kernel oil, and mixtures thereof. In some embodiments, the hard vegetable butter may be selected from the group consisting of cocoa butter, illipe butter, murumuru butter, kokum butter, coconut oil, and mixtures thereof.

In some preferred embodiments, the hard vegetable butter comprises as least cocoa butter. In some preferred embodiments, the hard vegetable butter is cocoa butter.

In some embodiments, the hard vegetable butter (e.g. cocoa butter) may be present in an amount of from about 5% to about 35% by weight of the solid cosmetic composition.

In some embodiments, the hard vegetable butter (e.g. cocoa butter) may be present in an amount of from about 10% to about 35% by weight of the solid cosmetic composition, such as from about 15% to about 30% by weight of the solid cosmetic composition, such as from about 20% to about 30% by weight of the solid cosmetic composition.

In some preferred embodiments, the hard vegetable butter (e.g. cocoa butter) may be present in an amount of from about 15% to about 35% by weight of the solid cosmetic composition, preferably from about 20% to about 30% by weight of the solid cosmetic composition, and more preferably from about 25% to about 30% by weight of the solid cosmetic composition.

In some embodiments, the solid cosmetic composition according to this aspect comprises:
(i) henna in an amount of from about 5% to about 75% by weight of the solid cosmetic composition;
(ii) a hard vegetable butter (e.g. cocoa butter) in an amount of from about 15% to about 35% by weight of the solid cosmetic composition; and
(iii) at least one of noni fruit and hibiscus in an amount of from about 0.1% to about 15% by weight of the solid cosmetic composition.
   In some embodiments, the solid cosmetic composition according to this aspect comprises:
(i) henna in an amount of from about 5% to about 75% by weight of the solid cosmetic composition;
(ii) a hard vegetable butter (e.g. cocoa butter) in an amount of from about 25% to about 30% by weight of the solid cosmetic composition; and
(iii) at least one of noni fruit and hibiscus in an amount of from about 0.1% to about 15% by weight of the solid cosmetic composition.
   In some embodiments, the solid cosmetic composition according to this aspect comprises:
(i) henna in an amount of from about 15% to about 65% by weight of the solid cosmetic composition;
(ii) a hard vegetable butter (e.g. cocoa butter) in an amount of from about 15% to about 35% by weight of the solid cosmetic composition, such as from about 20% to about 30% by weight of the solid cosmetic composition;
(iii) a herbal dye material selected from the group consisting of hibiscus, noni fruit, and combinations thereof, preferably wherein the total amount of hibiscus and/or noni fruit is from about 0.1% to about 15% by weight of the solid cosmetic composition; and
(iv) at least one additional herbal dye material selected from the group consisting of indigo, Irish moss, nettle, coffee (e.g. ground coffee), rhubarb root, rosemary, chamomile, cassia obtava, spikenard, and mixtures thereof, preferably wherein the total amount of additional herbal dye material is from about 0.1% to about 50% by weight of the solid cosmetic composition.
   In some embodiments, the solid cosmetic composition according to this aspect consists essentially of or consists of:
(i) henna:
(ii) a hard vegetable butter (e.g. cocoa butter):
(iii) a herbal dye material selected from the group consisting of hibiscus, noni fruit, and combinations thereof:
(iv) at least one additional herbal dye material selected from the group consisting of indigo, Irish moss, nettle, coffee (e.g. ground coffee), rhubarb root, rosemary, chamomile, cassia obtava, spikenard, and mixtures thereof;
(v) a fruit juice, preferably selected from the group consisting of lemon juice, noni fruit juice, and mixtures thereof; and
optionally (vi) a fragrance and/or salt.
   In such embodiments, where the composition includes noni fruit powder as the herbal dye material, the composition further comprises noni fruit juice and salt (e.g. sea salt). In such embodiments, where the composition includes hibiscus powder as the herbal dye material, the composition further comprises lemon juice.
   In some embodiments, the solid cosmetic composition according to this aspect consists essentially of or consists of:
(i) henna;
(ii) a hard vegetable butter (e.g. cocoa butter);
(iii) noni fruit powder;
(iv) at least one additional herbal dye material selected from the group consisting of indigo, Irish moss, nettle, coffee (e.g. ground coffee), rhubarb root, rosemary, chamomile, cassia obtava, spikenard, and mixtures thereof;
(v) noni fruit juice;
(vi) a fragrance; and
(vii) salt, preferably sea salt.
   In some embodiments, the solid cosmetic composition according to this aspect consists essentially of or consists of:
(i) henna;
(ii) a hard vegetable butter (e.g. cocoa butter);
(iii) hibiscus powder;
(iv) at least one additional herbal dye material selected from the group consisting of indigo, Irish moss, nettle, coffee (e.g. ground coffee), rhubarb root, rosemary, chamomile, cassia obtava, spikenard, and mixtures thereof;
(v) lemon juice; and
(vi) a fragrance.
   In some embodiments, the solid cosmetic composition according to this aspect consists essentially of:
(i) henna in an amount of from about 15% to about 65% by weight of the solid cosmetic composition, such as from about 25% to about 65% by weight of the solid cosmetic composition;
(ii) a hard vegetable butter (e.g. cocoa butter) in an amount of from about 25% to about 30% by weight of the solid cosmetic composition;
(iii) hibiscus (e.g. hibiscus powder), preferably in an amount of from about 1% to about 10% by weight of the solid cosmetic composition;
(iv) at least one additional herbal dye material selected from the group consisting of indigo, Irish moss, nettle, coffee (e.g. ground coffee), rhubarb root, rosemary, chamomile, cassia obtava, spikenard, and mixtures thereof, preferably wherein the total amount of additional herbal dye material is from about 0.1% to about 50% by weight of the solid cosmetic composition;
(v) lemon juice, preferably in an amount of from about 1% to about 5% by weight of the solid cosmetic composition; and
optionally (vi) a fragrance, optionally in an amount of from about 0.1% to about 1% by weight of the solid cosmetic composition.
   In some embodiments, the solid cosmetic composition according to this aspect consists of:
(i) henna in an amount of from about 15% to about 65% by weight of the solid cosmetic composition, such as from about 25% to about 65% by weight of the solid cosmetic composition;
(ii) a hard vegetable butter (e.g. cocoa butter) in an amount of from about 25% to about 30% by weight of the solid cosmetic composition;
(iii) hibiscus (e.g. hibiscus powder), preferably in an amount of from about 1% to about 10% by weight of the solid cosmetic composition;
(iv) at least one additional herbal dye material selected from the group consisting of indigo, Irish moss, nettle, coffee (e.g. ground coffee), rhubarb root, rosemary, chamomile, cassia obtava, spikenard, and mixtures thereof, preferably wherein the total amount of additional herbal dye material is from about 0.1% to about 50% by weight of the solid cosmetic composition, such as from about 0.1% to about 35% by weight of the solid cosmetic composition;
(v) lemon juice, preferably in an amount of from about 1% to about 5% by weight of the solid cosmetic composition; and
optionally (vi) a fragrance, optionally in an amount of from about 0.1% to about 1% by weight of the solid cosmetic composition.
   In some embodiments, the solid cosmetic composition according to this aspect consists essentially of:
(i) henna in an amount of from about 15% to about 65% by weight of the solid cosmetic composition, such as from about 15% to about 30% by weight of the solid cosmetic composition;
(ii) a hard vegetable butter (e.g. cocoa butter) in an amount of from about 25% to about 30% by weight of the solid cosmetic composition;
(iii) noni fruit (e.g. noni fruit powder and/or noni fruit juice), preferably in an amount of from about 1% to about 10% by weight of the solid cosmetic composition;
(iv) at least one additional herbal dye material selected from the group consisting of indigo, Irish moss, nettle, coffee (e.g. ground coffee), rhubarb root, rosemary, chamomile, cassia obtava, spikenard, and mixtures thereof, preferably wherein the total amount of additional herbal dye material is from about 0.1% to about 50% by weight of the solid cosmetic composition, such as from about 30% to about 50% by weight of the solid cosmetic composition;
(v) salt, preferably in an amount of from about 1% to about 5% by weight of the solid cosmetic composition; and optionally (vi) a fragrance, optionally in an amount of from about 0.1% to about 1% by weight of the solid cosmetic composition.
   In some embodiments, the solid cosmetic composition according to this aspect consists essentially of:
(i) henna in an amount of from about 15% to about 65% by weight of the solid cosmetic composition, such as from about 15% to about 30% by weight of the solid cosmetic composition;
(ii) a hard vegetable butter (e.g. cocoa butter) in an amount of from about 25% to about 30% by weight of the solid cosmetic composition;
(iii) noni fruit powder and noni fruit juice, preferably wherein the noni fruit powder is present in an amount of from about 0.5% to about 5% by weight of the solid cosmetic composition, and preferably wherein the noni fruit juice is present in an amount of from about 0.5% to about 5% by weight of the solid cosmetic composition;
(iv) at least one additional herbal dye material selected from the group consisting of indigo, Irish moss, nettle, coffee (e.g. ground coffee), rhubarb root, rosemary, chamomile, cassia obtava, spikenard, and mixtures thereof, preferably wherein the total amount of additional herbal dye material is from about 0.1% to about 50% by weight of the solid cosmetic composition, such as from about 30% to about 50% by weight of the solid cosmetic composition;
(v) salt, preferably in an amount of from about 1% to about 5% by weight of the solid cosmetic composition; and
optionally (vi) a fragrance, optionally in an amount of from about 0.1% to about 1% by weight of the solid cosmetic composition.
   In some embodiments, the solid cosmetic composition according to this aspect consists of:
(i) henna in an amount of from about 15% to about 65% by weight of the solid cosmetic composition, such as from about 15% to about 30% by weight of the solid cosmetic composition;
(ii) a hard vegetable butter (e.g. cocoa butter) in an amount of from about 25% to about 30% by weight of the solid cosmetic composition;
(iii) noni fruit (e.g. noni fruit powder and/or noni fruit juice), preferably in an amount of from about 1% to about 10% by weight of the solid cosmetic composition;
(iv) at least one additional herbal dye material selected from the group consisting of indigo, Irish moss, nettle, coffee (e.g. ground coffee), rhubarb root, rosemary, chamomile, cassia obtava, spikenard, and mixtures thereof, preferably wherein the total amount of additional herbal dye material is from about 0.1% to about 50% by weight of the solid cosmetic composition, such as from about 30% to about 50% by weight of the solid cosmetic composition;
(v) salt, preferably in an amount of from about 1% to about 5% by weight of the solid cosmetic composition; and
optionally (vi) a fragrance, optionally in an amount of from about 0.1% to about 1% by weight of the solid cosmetic composition.
   In some embodiments, the solid cosmetic composition according to this aspect consists essentially of:
(i) henna in an amount of from about 15% to about 65% by weight of the solid cosmetic composition, such as from about 15% to about 30% by weight of the solid cosmetic composition;
(ii) a hard vegetable butter (e.g. cocoa butter) in an amount of from about 25% to about 30% by weight of the solid cosmetic composition;
(iii) noni fruit powder and noni fruit juice, preferably wherein the noni fruit powder is present in an amount of from about 0.5% to about 5% by weight of the solid cosmetic composition, and preferably wherein the noni fruit juice is present in an amount of from about 0.5% to about 5% by weight of the solid cosmetic composition;
(iv) at least one additional herbal dye material selected from the group consisting of indigo, Irish moss, nettle, coffee (e.g. ground coffee), rhubarb root, rosemary, chamomile, cassia obtava, spikenard, and mixtures thereof, preferably wherein the total amount of additional herbal dye material is from about 0.1% to about 50% by weight of the solid cosmetic composition, such as from about 30% to about 50% by weight of the solid cosmetic composition;
(v) salt, preferably in an amount of from about 1% to about 5% by weight of the solid cosmetic composition; and
optionally (vi) a fragrance, optionally in an amount of from about 0.1% to about 1% by weight of the solid cosmetic composition.
   A solid cosmetic composition according to this aspect may also include a polyol. The polyol may be as described hereinabove, and all embodiments noted above may equally apply to this aspect. The polyol may preferably be glycerol.
   Thus, in some embodiments, the solid cosmetic composition comprises:
(i) henna;
(ii) a hard vegetable butter;
(iii) a herbal dye material selected from the group consisting of hibiscus, noni fruit, and combinations thereof; and
(iv) a polyol.
   In some embodiments, the solid cosmetic composition comprises:
(i) henna;
(ii) cocoa butter;
(iii) a herbal dye material selected from the group consisting of hibiscus, noni fruit, and combinations thereof; and
(iv) glycerol.
   Thus, in some embodiments, the solid cosmetic composition comprises:
(i) henna;
(ii) a hard vegetable butter;
(iii) a herbal dye material selected from the group consisting of hibiscus powder, noni fruit powder, and combinations thereof; and
(iv) a polyol.
   In some embodiments, the solid cosmetic composition comprises:
(i) henna;
(ii) cocoa butter;
(iii) a herbal dye material selected from the group consisting of hibiscus powder, noni fruit powder, and combinations thereof; and
(iv) glycerol.
   In some embodiments, the solid cosmetic composition comprises:
(i) henna in an amount of from about 5% to about 75% by weight of the solid cosmetic composition;
(ii) a hard vegetable butter in an amount of from about 5% to about 35% by weight of the solid cosmetic composition;
(iii) a herbal dye material selected from the group consisting of hibiscus (e.g. powder), noni fruit (e.g. powder), and combinations thereof; and.
(iv) a polyol in an amount of from about 1% to about 35% by weight of the solid cosmetic composition.

In some preferred embodiments, the solid cosmetic composition according to this aspect is substantially free from a polyol (e.g. glycerol). Preferably, the solid cosmetic composition according to this aspect is free from (i.e. includes 0%) of a polyol, such as glycerol.

In some embodiments, the solid cosmetic composition further comprises one or more cosmetically acceptable additives of the type as described hereinabove. The one or more cosmetically acceptable additives may be present in amounts as described hereinabove.

The solid cosmetic composition according to this further aspect may be prepared according to any suitable process. In some embodiments, the process for the production of a solid cosmetic composition as defined herein comprises the steps of:
(a) combining:
   (i) henna,
   (ii) a hard vegetable butter, and
   (iii) a herbal dye material selected from the group consisting of hibiscus, noni fruit, and combinations thereof; and
(b) allowing the resultant combination to solidify.

In some embodiments, hard vegetable butter (e.g. cocoa butter) is first heated to melt the butter. The henna and powdered herbal dye materials may then be added and mixed with the vegetable butter.

In some embodiments, the composition comprises a fruit juice, such as lemon juice and/or noni fruit juice. In such embodiments, the process comprises a step of filtering the fruit juice and then adding the fruit juice (optionally in combination with a fragrance) to the mixture of powdered herbal dye materials, henna and vegetable butter, and mixed.

All disclosure of the process for preparing a composition according to the first aspect equally applies to the above two further aspects.

### Method

As described herein, there is provided a cosmetic method comprising contacting the hair of a user with a solid cosmetic composition as defined herein.

Although the invention is primarily for use on hair (and in particular as a dye for colouring hair), it may also be used on the skin or other materials. Therefore, in one aspect, there is also provided a cosmetic method comprising contacting the hair or skin of a user with a solid cosmetic composition as defined herein.

As will be appreciated by the skilled person, in view of the solid properties of the present invention, the user may contact the skin or hair directly with the solid cosmetic composition. There may be no need to dispense the product from within some external packaging prior to application to the skin, as is the case for conventional liquid hair dyes or colourants.

Preferably, the solid cosmetic composition may be contacted with the hair of a user via a method comprising:
(i) contacting the solid cosmetic composition with water,
(ii) heating the water to melt the solid cosmetic composition and to provide a semi-solid (e.g. gel-like) or viscous liquid,
(iii) applying the semi-solid or liquid substance to the hair of a user.

For example, to use the product of the present invention, the end user will typically place the product in a container. A small amount of warm or hot water can then be added to the container, typically to just cover the solid product. The product will start to disperse. Once a significant amount of product has dispersed more water can be added and the product gently stirred. Water can then be added until the desired consistency of dispersed product has been formed. This consistency may be determined by the user based on, for example, their preference or their hair type or condition.

Before applying water to the product, the product may be broken up into pieces. For example, the composition may be in the form of a bar or brick, from which a dose to be applied to the hair is snapped or broken off. It has been surprisingly found that the combination of a hard vegetable butter and polyol (e.g. glycerol) provides a solid product that is easier to break apart into pieces which may be subsequently melted such that the product can be applied to the hair than a product that contains a high level of cocoa butter. It has also been found that the product may melt more quickly when added to heated water.

Once applied to the hair, the product may be left in the hair for at least 30 minutes, and preferably at least an hour and preferably no more than 2 hours. The hair may subsequently be rinsed.

### EXAMPLES

The invention will now be described with reference to the following non-limiting examples.

### Example 1

A product having the following composition is prepared:

Batch Size (g): 100.00

| | Formula | |
|---|---|---|
| Component | (% by weight) | |
| Cocoa Butter | 12 | 12.00 |
| Glycerol | 16 | 16.00 |
| Red henna powder | 24 | 24.00 |
| Indigo powder | 35 | 35.00 |
| Irish moss powder | 0.5 | 0.50 |
| Noni fruit powder | 4 | 4.00 |
| Sea salt | 2.5 | 2.50 |
| Noni juice | 5 | 5.00 |
| Fragrance | 1 | 1.00 |
| TOTAL | 100 | 100.00 |

The product is prepared using the following method:
1. The cocoa butter is melted via heating, and then the glycerol is added and mixed with the melted cocoa butter.
2. The herbal dye powders and henna powder are mixed together.
3. Some of the powder mixture is added to the mixture of butter and glycerol, and the combination mixed. The powder mixture is then continuously and slowly added whilst mixing.
4. The noni juice, salt and fragrance is added when all of the powder has been mixed through, and the combination mixed quickly and thoroughly.
5. The resulting mixture is pressed into moulds while still warm.
6. The mixture is left in the moulds for around 12 hours to 72 hours to set.

### Example 2

A product having the following composition is prepared:

Batch Size (g): 100.00

| | Formula | |
|---|---|---|
| Component | (% by weight) | |
| Cocoa Butter | 12 | 12.00 |
| Glycerol | 16 | 16.00 |
| Red henna powder | 55 | 55.00 |
| Rosemary powder | 1 | 1.00 |
| Hibiscus flower powder | 10 | 10.00 |
| Lemon juice | 5 | 5.00 |
| Fragrance | 1 | 1.00 |
| TOTAL | 100 | 100.00 |

The product is prepared using the following method:
1. The cocoa butter is melted via heating, and then the glycerol is added and mixed with the melted cocoa butter.
2. The herbal dye powders and henna powder are mixed together.
3. Some of the powder mixture is added to the mixture of butter and glycerol, and the combination mixed. The powder mixture is then continuously and slowly added whilst mixing.
4. The lemon juice and fragrance is added when all of the powder has been mixed through, and the combination mixed quickly and thoroughly.
5. The resulting mixture is pressed into moulds while still warm.
6. The mixture is left in the moulds for around 12 hours to 72 hours to set.

### Example 3

A product having the following composition is prepared:

Batch Size (g): 100.00

| | Formula | |
|---|---|---|
| Component | (% by weight) | |
| Cocoa Butter | 30 | 30.00 |
| Red henna powder | 22 | 22.00 |
| Indigo powder | 35 | 35.00 |
| Irish moss powder | 0.5 | 0.50 |
| Noni fruit powder | 4 | 4.00 |
| Sea salt | 2.5 | 2.50 |
| Noni juice | 5 | 5.00 |
| Fragrance | 1 | 1.00 |
| TOTAL | 100 | 100.00 |

The product is prepared using the following method:
1. The cocoa butter is melted via heating.
2. The herbal dye powders and henna powder are mixed together.
3. Some of the powder mixture is added to the heated butter, and the combination mixed. The powder mixture is then continuously and slowly added whilst mixing. The mixture is blended until thick.
4. The noni juice, salt and fragrance is added when all of the powder has been mixed through, and the combination mixed quickly and thoroughly.
5. The resulting mixture is pressed into moulds while still warm.
6. The mixture is left in the moulds for around 12 hours to 48 hours to set.

### Example 4

A product having the following composition is prepared:

Batch Size (g): 100.00

| | Formula | |
|---|---|---|
| Component | (% by weight) | |
| Cocoa Butter | 27 | 27.00 |
| Red henna powder | 56 | 56.00 |
| Rosemary powder | 1 | 1.00 |
| Hibiscus flower powder | 10 | 10.00 |
| Lemon juice | 5 | 5.00 |
| Fragrance | 1 | 1.00 |
| TOTAL | 100 | 100.00 |

The product is prepared using the following method:
1. The cocoa butter is melted via heating.
2. The herbal dye powders and henna powder are mixed together.
3. Some of the powder mixture is added to the heated butter, and the combination mixed. The powder mixture is then continuously and slowly added whilst mixing. The mixture is blended until thick.
4. The lemon juice and fragrance is added when all of the powder has been mixed through, and the combination mixed quickly and thoroughly.
5. The resulting mixture is pressed into moulds while still warm.
6. The mixture is left in the moulds for around 12 hours to 48 hours to set.

### Example 5

A solid product was prepared in accordance with the present invention. The product included the following ingredients:
Red henna powder
Glycerol
Cocoa butter
Indigo powder
Hibiscus flower powder
Ground coffee
Lemon juice
Fragrance
Irish Moss powder

The product according to Example 5 was compared with a commercial product—Marron Henna Brick sold by Lush Limited. The Marron Henna Brick includes the same ingredients as those noted above, except that the Marron Brick does not include any glycerol nor any hibiscus powder.

Each of the products according to Example 5 were provided to a panel of 20 independent testers in order to test each of the samples. 8 of these testers had the product applied for them (in accordance with the method outlined below) and therefore did not prepare or apply the product themselves. Each of the remaining 12 testers was asked to apply the product to their hair using the following method:
1. Break up the brick and gradually stir in freshly boiled water.
2. Add Ultrabland or Ultraplant to the hairline and around the ears.
3. Comb and section the hair, check the henna product is a comfortable temperature, and apply to the roots of the hair, working from the nape of the neck up to the crown. Use a brush for extra precision.
4. Smear abundantly through the lengths and ends of the hair with (gloved) hands and pile on top of the head.
5. Cover with a shower cap for a warmer result, leaving on for up to two hours.
6. Rinse thoroughly.

Each tester was then asked to provide feedback on the product compared with the Marron Henna Brick.

The testers were asked the following questions:

Was this Henna brick easier to prepare than the previous Henna?

The 8 testers who had the henna applied for them were not asked this question
12 out of 12 testers replied with “Yes”

Response comments included:
“The henna brick was softer and easier to break/cut up, and it dissolved quicker.”
“Much easier to cut, and took less time to melt than usual, did not get a skin over it either.”
   Was this Henna brick easier to apply than the previous Henna?

Of the 20 testers, 4 of those who had someone else apply it for them declined to answer
8 out of the remaining 16 testers replied with “Yes” (3 replied with “The same”)

Response comments included:
“Much smoother consistency, no solid chunks when applying.”
   Was the resulting shade improved compared to the previous time you Henna'd your hair?

14 out of 20 testers replied with “Yes” (3 testers replied with “The same”)

### Example 6

A solid product was prepared in accordance with the present invention. The product included the following ingredients:
Indigo powder
Red henna powder
Glycerol
Cocoa butter
Noni fruit powder
Noni fruit juice
Sea salt
Fragrance
Irish Moss powder

The product according to Example 6 was compared with the comparative products 1 and 2. Comparative product 1 was identical to the product of Example 6, but where the glycerol was replaced with more cocoa butter. Comparative product 2 was identical to the product of Example 6, but where glycerol was replaced with almond oil. Neither comparative product contained any glycerol or other polyol.

Each of the products were formulated and applied to blonde human hair using the method referred to in Example 5 above.

Comparative product 1 including only cocoa butter was in the form of a solid, but ‘blooming’ occurred, whereby the surface of the product was found to turn patchy/spotty where it had oxidised. This created an undesirable appearance, which was considered to be off-putting for the user. In contrast, the appearance of the product in accordance with Example 6 had a smooth finish and the product was in the form of a solid that did not crack or crumble.

The product according to Example 6 was found to provide the most desirable product in terms of a solid brick that provides the most depth and the coolest resulting shade when applied to the hair.

When applied to the hair, comparative product 1 dyed the hair in such a way that the result was a warmer (less desirable) appearance of the hair than was achieved by the product of Example 6 including cocoa butter and glycerol.

When applied to the hair, comparative product 2 including almond oil dyed the hair in such a way that a patchy result with uneven coverage was provided. Dark spots were formed on the hair.

### Example 7

Solid products were prepared in accordance with the present invention and tested by consumers. Each of the products included henna, cocoa butter and at least one of noni fruit and hibiscus powder.

The solid products were tested by a panel of testers using the method referred to in Example 5 above.

A number of the testers were users of previous commercial henna products. The testers were asked to compare the products according to the present invention with those previous commercial henna products. Of the testers who had used previous commercial henna products, the majority said that the new products were the same or better than the previous products in certain respects. Some found the new products easier to apply; some found that the colour was improved compared to previous products.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the claims.

1. A solid cosmetic composition comprising:
(i) henna in an amount of from about 5% to about 75% by weight of the solid cosmetic composition;
(ii) a hard vegetable butter in an amount of from about 5% to about 35% by weight of the solid cosmetic composition; and
(iii) a polyol in an amount of from about 1% to about 35% by weight of the solid cosmetic composition.

2. The solid cosmetic composition according to claim 1, wherein the henna is present in an amount of from about 15% to about 65% by weight of the solid cosmetic composition.

3. The solid cosmetic composition according to claim 1, wherein the hard vegetable butter is present in an amount of from about 5% to about 20% by weight of the solid cosmetic composition.

4. The solid cosmetic composition according to claim 1, wherein the hard vegetable butter is selected from the group consisting of cocoa butter, illipe butter, murumuru butter, kokum butter, coconut oil, palm kernel oil, and mixtures thereof.

5. The solid cosmetic composition according to claim 1, wherein the hard vegetable butter is cocoa butter.

6. The solid cosmetic composition according to claim 1, wherein the polyol is present in an amount of from about 10% to about 20% by weight of the solid cosmetic composition.

7. The solid cosmetic composition according to claim 1, wherein the polyol is selected from the group consisting of glycerol, propylene glycol, propan-1,3-diol, sorbitol, xylitol, isomalt, erythritol, lactitol, mannitol, maltitol, and mixtures thereof.

8. The solid cosmetic composition according to claim 1, wherein the polyol is glycerol.

9. The solid cosmetic composition according to claim 1, wherein the weight ratio of hard vegetable butter to polyol is from about 4:1 to about 1:4.

10. The solid cosmetic composition according to claim 1, further comprising a herbal dye material, wherein the herbal dye material is other than henna.

11. The solid cosmetic composition according to claim 10, wherein the herbal dye material is selected from the group consisting of indigo, Irish moss, nettle, ground coffee, noni fruit, hibiscus, rhubarb root, chamomile, cassia obtava, rosemary, spikenard, and mixtures thereof.

12. The solid cosmetic composition according to claim 10, wherein the herbal dye material is present in an amount of from about 5% to about 60% by weight of the solid cosmetic composition.

13. The solid cosmetic composition according to claim 10, wherein a total combined amount of henna and herbal dye material is from about 40% to about 80% by weight of the solid cosmetic composition.

14. The solid cosmetic composition according to claim 10, wherein the herbal dye material comprises noni fruit powder, hibiscus powder or combinations thereof.

15. The solid cosmetic composition according to claim 14, wherein the herbal dye material is or comprises hibiscus powder present in an amount of from about 0.1% to about 15% by weight of the solid cosmetic composition.

16. The solid cosmetic composition according to claim 14, wherein the herbal dye material is or comprises noni fruit powder present in an amount of from about 0.1% to about 15% by weight of the solid cosmetic composition.

17. The solid cosmetic composition according to claim 1, further comprising fruit juice.

18. The solid cosmetic composition according to claim 17, wherein the fruit juice is selected from the group consisting of lemon juice, noni fruit juice, lime juice, grapefruit juice, yuzu juice, and mixtures thereof.

19. The solid cosmetic composition according to claim 17, wherein the fruit juice is present in an amount of from about 0.1% to about 8% by weight of the solid cosmetic composition.

20. The solid cosmetic composition according to claim 1, further comprising one or more cosmetically acceptable additives selected from a binder, filler, exfoliating material, essential oil, vitamin, perfume, fragrance, colouring agent, decorative item, effervescent component, surfactant, humectant, conditioning agent, acidifying agent, a base, and mixtures thereof.

21. A process for the production of a solid cosmetic composition as defined in claim 1, the process comprising the steps of:
(a) combining:
(i) henna,
(ii) a hard vegetable butter, and
(iii) a polyol; and
(b) allowing the resultant combination to solidify.

22. The process according to claim 21, wherein the step (b) comprises introducing the resultant combination into a mould before allowing said combination to solidify.

23. The process according to claim 21, wherein the process further comprises adding fruit juice, herbal dye material in powdered form or combinations thereof before, during or after step (a).

24. A solid cosmetic composition obtained or obtainable by the process of claim 21.

25. A solid cosmetic composition comprising (i) henna; (ii) a hard vegetable butter; and (iii) a herbal dye material selected from the group consisting of hibiscus, noni fruit, amla, Bhrinhraj/maka (Eclipta alba or Eclipta prostrata), spikenard, Kuth root, Valerian root, alkanet, blueberry, pomegranate, and combinations thereof.

26. A solid cosmetic composition comprising:
(i) henna;
(ii) a hard vegetable butter; and
(iii) a herbal dye material selected from the group consisting of hibiscus, noni fruit, and combinations thereof.

27. The solid cosmetic composition according to claim 25, wherein the hard vegetable butter comprises cocoa butter.

28. The solid cosmetic composition according to claim 25, wherein the hard vegetable butter is present in an amount of from about 15% to about 35% by weight of the solid cosmetic composition.

29. The solid cosmetic composition according to claim 25, wherein the composition comprises:
(i) the henna in an amount of from about 5% to about 75% by weight of the solid cosmetic composition;
(ii) the hard vegetable butter in an amount of from about 15% to about 35% by weight of the solid cosmetic composition; and
(iii) the herbal dye material selected from the group consisting of hibiscus, noni fruit, and combinations thereof, wherein the total amount of noni fruit and hibiscus is from about 0.1% to about 15% by weight of the solid cosmetic composition.

30. The solid cosmetic composition according to claim 25, wherein the herbal dye material is or comprises hibiscus powder present in an amount of from about 0.1% to about 15% by weight of the solid cosmetic composition.

31. The solid cosmetic composition according to claim 25, wherein the herbal dye material is or comprises noni fruit powder present in an amount of from about 0.1% to about 15% by weight of the solid cosmetic composition.

32. The solid cosmetic composition according to claim 25, further comprising fruit juice, wherein the fruit juice is selected from the group consisting of lemon juice, noni fruit juice, lime juice, grapefruit juice, yuzu juice, and mixtures thereof.

33. The solid cosmetic composition according to claim 32, wherein the fruit juice is present in an amount of from about 0.1% to about 8% by weight of the solid cosmetic composition.

34. The solid cosmetic composition according to claim 25, further comprising one or more cosmetically acceptable additives selected from a binder, filler, exfoliating material, essential oil, vitamin, perfume, fragrance, colouring agent, decorative item, effervescent component, surfactant, humectant, conditioning agent, acidifying agent, a base, and mixtures thereof.

35. The solid cosmetic composition according to claim 26, wherein the composition consists essentially of:
(i) the henna;
(ii) the hard vegetable butter;
(iii) the herbal dye material selected from the group consisting of hibiscus, noni fruit, and combinations thereof;
(iv) at least one additional herbal dye material selected from the group consisting of indigo, Irish moss, nettle, coffee, rhubarb root, rosemary, chamomile, cassia obtava, spikenard, and mixtures thereof;
(v) a fruit juice, selected from the group consisting of lemon juice, noni fruit juice, and mixtures thereof; and
(vi) a fragrance and/or salt.

36. The solid cosmetic composition according to claim 26, wherein the composition consists essentially of:
(i) the henna;
(ii) the hard vegetable butter;
(iii) noni fruit powder;
(iv) at least one additional herbal dye material selected from the group consisting of indigo, Irish moss, nettle, coffee, rhubarb root, rosemary, chamomile, cassia obtava, spikenard, and mixtures thereof;
(v) noni fruit juice;
(vi) a fragrance; and
(vii) salt.

37. The solid cosmetic composition according to claim 25, wherein the composition consists essentially of:
(i) the henna;
(ii) the hard vegetable butter;
(iii) hibiscus powder,
(iv) at least one additional herbal dye material selected from the group consisting of indigo, Irish moss, nettle, coffee, rhubarb root, rosemary, chamomile, cassia obtava, spikenard, and mixtures thereof,
(v) lemon juice; and
(vi) a fragrance.

38. A cosmetic method comprising contacting the hair of a user with the solid cosmetic composition as defined in claim 1.
